(19) United States
(12) Patent Application Publication
(10) Pub. No.: US 2026/0176628 A1
(43) Pub. Date: Jun. 25, 2026

(54) TARGETED EPIGENETIC EDITING AS NOVEL THERAPY FOR MALIGNANT GLIOMA

(71) Applicant: The Regents of the University of California, Oakland, CA (US)

(72) Inventors: Tie Li, Harbor City, CA (US); Albert Lai, Glendale, CA (US); Serendipity Zapanta Rinonos, Gainesville, FL (US); Sean T. Pianka

(73) Assignee: The Regents of the University of California, Oakland, CA (US)

(21) Appl. No.: 19/128,348

(22) PCT Filed: Dec. 1, 2023
(86) PCT No.: PCT/US2023/082044

§ 371 Date: May 8, 2025

(30) Foreign Application Priority Data
Dec. 1, 2022 (US) 63429348

(51) Int. Cl. C12N 15/113; C12N 9/22

(52) U.S. Cl. CPC C12N 15/113; C12N 9/22; C12N 2310/20

(57) ABSTRACT

Malignant glioma has a poor prognosis, despite current therapeutic modalities. Standard of care therapy consists of surgical resection, fractionated radiotherapy concurrently administered with temozolomide (TMZ), a DNA-alkylating chemotherapeutic agent, followed by adjuvant cycles of TMZ. O6-methylguanine DNA methyltransferase (MGMT) is a DNA repair enzyme which removes alkylated lesions from tumor DNA, thereby promoting chemoresistance. MGMT promoter methylation is a known predictor of temozolomide responsiveness; patients with unmethylated MGMT have a poorer prognosis. We employed deactivated Cas9-CRISPR technology to effectively target methylation in the vicinity of the MGMT methylation-specific polymerase chain reaction (MSP) region, as mediated by the catalytic domain of DNA methyltransferase DNMT3A. In doing so, we discovered that the selected dCas9/CRISPR-mediated methylation of certain regions of MGMT gene sequences can be used to enhance chemosensitivity in malignant glioma cells.

### CROSS REFERENCE TO RELATED APPLICATIONS

This application claims the benefit under 35 U.S.C. Section 119 (e) of co-pending and commonly-assigned U.S. Provisional Patent Application No. 63/429,348, filed Dec. 1, 2022, entitled “TARGETED EPIGENETIC EDITING AS NOVEL THERAPY FOR MALIGNANT GLIOMA”, which application is incorporated by reference herein.

### STATEMENT REGARDING FEDERALLY SPONSORED RESEARCH AND DEVELOPMENT

This invention was made with government support under CA009056, and CA247525 awarded by the National Institutes of Health. The government has certain rights in the invention.

### TECHNICAL FIELD

Embodiments of the disclosure concern at least the fields of molecular biology, epigenetics, cell biology, oncology and medicine.

### BACKGROUND OF THE INVENTION

For malignant glioma, specifically glioblastoma (GBM), a central aspect of clinical decision-making and prognosis is the de novo methylation status of O6-methylguanine-DNA methyltransferase (MGMT) (1-8), routinely assayed during neuropathologic diagnosis. MGMT repairs the toxic DNA lesion O6-methylguanine induced by alkylating chemotherapeutic agents, such as temozolomide (TMZ), thereby undermining the mechanism of action, leading to chemoresistance. As the only FDA-approved drug with relative improvement in survival, TMZ comprises the current standard of care chemotherapy in combination with fractionated radiation (6, 7). Approximately 40% of GBM patients harbor methylated MGMT, silencing expression in tumor cells, enhancing chemosensitivity and survival (progression-free survival (PFS)=10.3 months, overall survival (OS)=21.7 months) (2). By contrast, the preponderance of GBM patients (60%) harbor unmethylated MGMT and exhibit chemoresistance to TMZ (1-3, 5-12), markedly reducing survival (PFS=5.3 months, OS=12.7 months) (2).

To date, multiple attempts to combat TMZ chemoresistance, via direct inhibition or cellular depletion, have yielded no significant clinical improvements. A Phase II clinical trial using the direct MGMT inhibitor O-6-benzylguanine showed limited benefits but lacked clinical feasibility due to severe dose-limiting toxicities (including off-target bone marrow suppression) (13, 14). A Phase III trial implemented dose-dense TMZ, aiming to deplete MGMT in tumor cells, but failed to improve TMZ sensitivity (15).

In view of the above, there is a need in the art for methods and materials that can increase the therapeutic efficacy of chemotherapeutic agents such as temozolomide.

### SUMMARY OF THE INVENTION

As discussed in detail below, we have discovered that the targeted methylation of selected CpG sites within a certain MGMT promoter region of human glioma cells make these cells sensitive to temozolomide. This discovery presents a novel treatment opportunity for patients suffering from malignant gliomas such as glioblastoma multiforme. In this context, embodiments of the invention disclosed herein harness discoveries of specific functional sites for epigenetic modification as a novel therapeutic strategy, one that involves the targeted conversion of selected MGMT promoter regions from an unmethylated to a methylated status, thereby enhancing temozolomide chemosensitivity in malignant gliomas.

The invention disclosed herein has a number of embodiments. Embodiments of the invention include, for example, compositions of matter comprising a CRISPR ribonucleotide complex; wherein the CRISPR ribonucleotide complex includes a fusion protein comprising a dCas9 polypeptide sequence fused with a human de novo DNA methyltransferase 3A polypeptide sequence such that the fusion protein methylates CpG sequences in the human genome and the fusion protein does not cleave DNA. Such compositions of matter also include at least one single guide RNA, wherein the single guide RNA comprises at least 17 nucleotides that are complementary to:

(SEQ ID NO: 1)
TGCCCCTCGGCCCCGCCCCCGCGCCCCGGATATGCTGGGACAGCCCGCGC
CCCTAGAACGCTTTGCGTCCCGACGCCCGCAGGTCCTCGCGGTGCGCACC
GTTTGCGACTTGGTGAGTGTCTGGGTCGCCTCGCTCCCGGAAGAGTGCGG
AGCTCTCCCTCGGGACGGTGGCAGCCTCGAGTGGTCCTGCAGGCGCCCTC
ACTTCGCCGTCGGGTGTGGGGCCGCCCTGACCCCCACCCATCCCGGGCG
A.

Embodiments of the invention also include methods of modulating the physiological response of a mammalian glioma cell to a chemotherapeutic agent. Typically these methods comprise combining a mammalian glioma cell with a composition of the invention disclosed herein so that the CRISPR ribonucleotide methylation complex methylates a plurality of CpG dinucleotide sites in a O6-methylguanine DNA methyltransferase polynucleotide in the genome of the mammalian glioma cell such that the physiological response of the mammalian glioma cell to the chemotherapeutic agent is modulated.

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 Schematic overview and verification of the dCas9/DNMT3A catalytic domain CRISPR-based methylation system in LN18 human glioma cells. (a) Schematic representation of the dCas9-DNMT3A-CD complex bound to a segment of DNA at the MGMT gene on chromosome 10. Deactivated Cas9 (dCas9) is unable to cut DNA and is fused to the DNMT3A catalytic domain. Single guide RNA (sgRNA) sequences (blue) can bind to complementary sequences within the genome, which permits dCas9 (teal) to be able to recognize and bind to DNA (purple). Once bound, DNMT3A-CD (magenta) can then induce methylation in CpG sites (represented as the “Me” labeled black circles) upstream of sgRNA complementary sequences. (b) Verification of dCas9-DNMT3A-CD protein expression in LN18 human glioma cells. Western blot images of HA-tagged dCas9-DNMT3A-CD fusion protein in LN18 cells sequentially transfected with pLVP-dCas9-DNMT3A-CD-V2 and pLenti-sgRNA-GFP (versus native cell line as negative control with no transfection constructs); representative blot shown here (from at least 3 replicate experiments). “NSC” indicates non-specific scrambled sgRNA transfection; “sgR1” and “sgR2” indicate replicate samples derived from cells with MGMT-sgRNA 1, 2, 3 and 4 transfection. Expected size of the dCas9/DNMT3A-CD fusion protein is approximately in the 200 kDa range, as shown, using anti-HA antibody-mediated detection. GAPDH served as the loading control. (c) Verification of sgRNA-GFP lentiviral transfection in LN18 human glioma cells. Representative fluorescent microscopic images (40× magnification) of the same LN18 cell lines in part (c) demonstrating GFP signal detection in cells transfected with GFP-tagged pLenti-sgRNA (LN18 SgR) vs. pLenti-NSC (scrambled sgRNA; LN18 NSC). DAPI shown as nuclear stain, with merged images in far-right column.

FIG. 2 Bisulfite sequencing of representative clones with induced methylation patterns of CpG sites at CRISPR-targeted regions within the MGMT CpG island. (a) Map of MGMT CpG island, 762 bp in length, encompassing promoter, exon 1, enhancer, and intron 1 regions. Comprehensive map of the MGMT gene with superimposed locations of Illumina probes (yellow boxes) as well as exon (salmon), intron (purple), and promoter/upstream (gray) regions. Locations of complementary sequences to the four sgRNAs are as shown. Open star, half-closed star and closed star regions indicate locations of differentially methylated Illumina probes. F1/R1 and F2/R2 indicate nested PCR primer pairs for Region 1 and Region 2, respectively. Inset: MGMT CpG island (individual CpG sites in light green) and relative locations of Region 1 and Region 2. CpG sites 22, 57, 72, and 96 indicate the specific sites flanking each region, numbered in order from 5′ to 3′ within the CpG island. Differentially methylated regions are shown (DMR1 and DMR2), located within assayed Region 1 and 2, respectively. MSP region is also shown within DMR2. Genetic regions and positions on Chromosome 10 were determined using the UCSC genome browser (GRCh37/hg19 assembly) and the 850K array probe annotation file provided by Illumina. (b) Lollipop schematic illustrating the distribution of CpG methylation sites in each representative clonal population (red circle=methylated site; gray circle=unmethylated site). Genomic DNA from LN18 cells containing either dCas9-DNMT3A/non-specific sgRNA or dCas9-DNMT3A/sgRNAs underwent sodium bisulfite treatment, followed by nested PCR amplification. Amplicons of Region 1 and Region 2 were obtained separately and used to generate individual clones for each region via TA cloning. Abbreviations for individual clone nomenclature (e.g., “4-NSC1” vs. “13-NSC1”): “4”=Region containing sgRNA4 target sequence; “13”=Region containing sgRNA 1, 2, 3 target sequences. “NSC” vs. “SgR” indicates whether clone source was transfected with non-specific sgRNA or sgRNA1, 2, 3, 4. Numbers following NSC or SgR designation indicate individual clone numbers. Each row of the schematic (per category, NSC or SgR) represents a single clone. Regional CpG sites as per Malley et al (2011): DMR1=CpG 25-50; DMR2=CpG 73-90; MSP=CpG 76-87 (MSP-F=CpG 76-80; MSP-R=CpG 84-87). Core/minimal promoter=CpG 50-62; Enhancer=CpG 82-87. (c) BISEQ chromatograms of representative clones, demonstrating methylation of target DNA sequences within the MGMT CpG island, derived from LN18 cells transfected with dCas9/DNMT3A-CD plus sgRNA constructs. Blue arrows indicate CpG sites that are methylated (converted from T-G to C-G). Region 1 includes the sequence targeted by sgRNA4 (s4). Region 2 includes the sequence targeted by sgRNA 1, 2 and 3 (s1, s2, s3).

FIG. 3 Expression of MGMT mRNA and protein in LN18 human glioma cells with CRISPR-mediated MGMT methylation via dCas9-DNMT3A-CD and MGMT-specific sgRNAs. (a) MGMT mRNA expression detected by RT-qPCR in LN18 cells expressing dCas9-DNMT3A CD/sgRNA targeted towards MGMT. Top: qPCR bar figure of expression mean+/−SEM (from a total of four repeated experiments). Bottom: DNA gel electrophoresis images of corresponding qPCR end products (from four separate experiments). Actin B (ACTB) served as the internal control. (b) Western blot analysis of MGMT expression levels in LN18 cells. GAPDH was used as the loading control. Representative results are shown here (from a total of four replicate experiments). Abbreviations: NSC=cells with non-specific scrambled sgRNA transfection; sg=cells with quadruple sgRNA 1, 2, 3 and 4 transfection. R1, R2=replicate samples.

FIG. 4 Effects of dCas9-DNMT3A-CD/MGMT-specific sgRNA targeted methylation on the sensitivity of glioma cells to temozolomide. MTT and clonogenic assays demonstrate effects of dCas9-DNMT3A-CD/sgRNA CRISPR-based methylation on the survival of LN18 glioma cells treated with TMZ in vitro. Negative control: LN18 NSC cells. The “n” indicates the number of separate experiments performed for each cell type. Results were normalized to the average negative control treatment condition (drug vehicle DMSO). TMZ concentrations ranged from 25 M to 100 M. Horizontal bars for all figures represent post hoc Tukey's multiple comparisons test results for groups of interest following two-way ANOVA (“ns” indicates “p>0.05”; * indicates p<0.05; ** indicates p<0.01; *** indicates p<0.001; **** indicates p<0.0001). (a) TMZ sensitivity measured via MTT survival assays in LN18 cells co-expressing dCas9-DNMT3A-CD (d3A) and MGMT-targeting sgRNAs (sg1, 2, 3, 4) compared to NSC lines. Differences were observed between sgRNA treated cells (ANOVA, F (1, 74)=213.3, p<0.0001) and applied TMZ concentration (ANOVA, F (2, 74)=93.52, p<0.0001); an interaction was also observed between sgRNA/NSC and TMZ (ANOVA, F (2, 74)=86.92, p<0.0001). MTT cell survival was calculated by subtracting 560 nm readings from 550 nm readings for TMZ-treated cells (25 M and 100 M, 5 days) versus DMSO treatment control condition, followed by normalization to the average control condition. (b) Effect of sgRNA methylation mediated TMZ sensitivity (ANOVA, F (1, 8)=134.9, p<0.0001) was comparable between dCas9-DNMT3A-CD and Cas9-mediated MGMT knockout lines with sgRNA1 and 2 (ANOVA, F (1, 8)=2.843, p=0.1303), when evaluated via MTT survival assays. (c) Clonogenic assay revealed TMZ sensitivity results comparable to MTT (ANOVA, F (2, 56)=186.2, p<0.0001). Two-way ANOVA results revealed an effect of sgRNA treatment (F (1, 56)=287.5, p<0.0001) as well as an interaction between TMZ sensitivity and sgRNA treatment (F (2, 56)=124.8, p<0.0001). (d) Clonogenic assay demonstrated similar results to (b) with increased TMZ sensitivity in dCas9-DNMT3A-CD and Cas9-mediated MGMT knockout lines (ANOVA, F (1, 8)=685.1, p<0.0001). Two-way ANOVA also showed a difference between the dCas9 and Cas9 systems (F (1, 8)=9.587, p=0.0147) as well as an interaction between system and TMZ sensitivity (F (1, 8)=9.587, p=0.0147).

FIG. 5 Validation of CRISPR-based dCas9-DNMT3A-CD targeted MGMT hypermethylation and differential RNA expression via correlation of Illumina EPIC 850k methylation array and RNA-Seq analysis. (a) Overview of the Illumina pipeline and generation of supervised hierarchical heatmaps. Raw data from the Illumina array (.idat files) were imported into R and matched to the Illumina annotation manifest by probe. Methylation values by probe were passed through a quality control check, CpG sites with single nucleotide polymorphisms (SNPs) were removed, and the data was normalized. These data were then clustered for methylation state by sample and probe ID using M-values with high variance (>2.5 SDs), for unsupervised heatmap generation. Probes were further isolated by adjusted p-value<0.05 (linear fit and eBayes analysis) for supervised heatmap generation. “On Target” and “Off Target” probes were identified by further filtering the supervised differential methylation data by M-values with high variance across samples (>2 SDs) and a low control average M-value (<−1) and high SgR average M-value (>−1). These genes were cross referenced with bulk RNA-Seq differential expression data (evaluated via DESeq2) to determine functionally significant “On Target” and “Off Target” hits. Each step shows a donut plot of approximate percentage of genes from the total array that emerged from the filter criteria for that step, with hypermethylation shown in red, hypomethylation shown in blue, and no change (under that criterion) shown in gray. (b) Unsupervised hierarchical clustering of M-values by Illumina probe (rows) and LN18 cell treatment (columns). M-value variance (standard deviation) across cell type for each probe was calculated for the entire Illumina 850K array and probes with the highest level of variance (2.5 SDs>average M-value SD; N=21,278) were isolated and plotted as a heatmap. Definition of nomenclature: R1, R2=replicates; t0=baseline harvest time point (corresponds to approximately 2 weeks after final lentiviral transfection, in this case, s/p GFP-sgRNA or GFP-NSC transfection); t2-harvested 2 months after t0; a=indicates samples run on 1st array batch; b=indicates samples run on 2nd array batch, subsequent to 1st array. (We performed 2 separate arrays, at different times, distinguished here by a and b.). (c) Raw M-value distributions for all Illumina probes and cell samples. Control LN18 NSC samples are shown by the blue (NSC-R1-t0-a, NSC-R1-t2-a, NSC-R1-t0-b) traces, while the LN18 SgR samples are shown by the magenta (SgR1-t0-a, SgR1-t2-a, SgR1-t0-b) and orange (SgR2-t0-a, SgR2-t2-a, SgR2-t0-b) traces. The approximate cutoff point for the first peak and “low methylation” threshold is indicated by the vertical gray line (−1). (d) Average trace of all control LN18 NSC samples, with the average M-value across all probes indicated by the vertical gray line. Vertical blue lines represent M-value standard deviations of varying degrees above and below this average. (e) Distribution of M-value variance for all Illumina probes across all samples, with summary statistics similar to panel (d) superimposed. These M-value distribution plots were used for establishing thresholds for determining large increases in methylation state between control LN18 NSC and SgR samples. (f) Following validation via unsupervised hierarchical clustering and generation of an initial supervised heatmap (refer to pipeline in (a) and Methods), we applied additional filtering for hierarchical clustering of all CpG island probes found to be differentially methylated according to the following criteria: 1) p-adj<0.05, 2) found in CpG island region, 3) exhibited an increase in methylation M-value from control LN18 NSC to SgR cells greater than 2 standard deviations above the average M-value variance (SD) in all cells/probes, and 4) contained an average control LN18 NSC methylation M-value of less than-1. Of these criteria, three probes within the MGMT gene were identified: cg12434587 (open star), cg01341123 (half-closed star), and cg12981137 (closed star), all of which were near sgRNA loci (see FIG. 1). (g) Of all the probes surveyed in (a), 333 unique genes were identified and intersected with DESeq2 differential expression bulk RNA-Seq data (Wald test, p-adj<0.05). Genes found in both data sets were deemed to be “On-Target and Off-Target” effects, which included MGMT and 9 other genes.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

Since its initial discovery in bacteria/archaea, the CRISPR (Clustered, Regularly Interspaced, Short Palindromic Repeats)/Cas9 system, with its mechanism of targeted expression silencing via nucleolytic cleavage of specific genetic sequences, has emerged as a viable and promising vehicle for genetic silencing applications within eukaryotic and mammalian contexts. One CRISPR system having a deactivated Cas9 (dCas9), with point mutations precluding DNA cleavage, retains localization specificity for target genes in combination with short guide RNAs. This system is a convenient tool to facilitate targeted epigenetic editing without causing permanent DNA cleavage. One such dCas9-based chimeric fusion with an epigenetic editor, the CpG methylator de novo DNA methyltransferase 3A (DNMT3A), has been developed; and an embodiment of this model employs the DNMT3A catalytic domain-only iteration (herein termed “DNMT3A-CD”) which we used for the studies disclosed below.

As discussed in detail below, we have discovered that the methylation of certain specific MGMT promoter region in glioma cells can be achieved via a dCas9/DNMT3A-CD/sgRNA CRISPR targeting system. Further studies determined that the selected methylation of certain regions in the MGMT promoter region in human glioma cells renders these cells more sensitive to temozolomide. Building upon these discoveries, we have developed a number of methods and materials that are designed to methylate selected MGMT promoter regions in glioma cells, thereby increasing the sensitivity of these cells to temozolomide.

Enhanced chemosensitivity achieved via epigenetic conversion of MGMT promoter status constitutes a direct demonstration of MGMT as a molecular driver in glioma pathophysiology. This has promising implications for future epigenetics-based clinical applications. Manipulating the epigenetic landscape of gliomas using targeted epigenetic editing represents a cutting-edge approach to probing fundamental cancer biology and revolutionizing therapeutic design. Recent advances in this field make it both possible and imperative that we develop tailored therapies for glioma patients who remain in acute and pressing need of new treatments. As disclosed herein, we discovered and harnessed targeted epigenetic editing approaches to induce methylation of the MGMT promoter region in order to downregulate the expression of MGMT in glioma model systems and achieve TMZ chemosensitization. Our novel approach to MGMT silencing represents a groundbreaking application of targeted molecular editing, namely, conversion to an epigenetic state known to enhance efficacy of standard-of-care therapies in malignant glioma. In summary, the current findings provide the initial foundation for future translational endeavors into the clinical arena.

The invention disclosed herein has a number of embodiments. Embodiments of the invention include, for example, compositions of matter comprising a CRISPR ribonucleotide complex; wherein the CRISPR ribonucleotide complex includes a fusion protein comprising a dCas9 polypeptide sequence fused with a human de novo DNA methyltransferase 3A polypeptide sequence such that the fusion protein methylates CpG sequences in the human genome and the fusion protein does not cleave DNA. Such compositions of matter include at least one single guide RNA, wherein the single guide RNA comprises at least 17 nucleotides that are complementary to:

(SEQ ID NO: 1)
TGCCCCTCGGCCCCGCCCCCGCGCCCCGGATATGCTGGGACAGCCCGCGC
CCCTAGAACGCTTTGCGTCCCGACGCCCGCAGGTCCTCGCGGTGCGCACC
GTTTGCGACTTGGTGAGTGTCTGGGTCGCCTCGCTCCCGGAAGAGTGCGG
AGCTCTCCCTCGGGACGGTGGCAGCCTCGAGTGGTCCTGCAGGCGCCCTC
ACTTCGCCGTCGGGTGTGGGGCCGCCCTGACCCCCACCCATCCCGGGCG
A.

In certain embodiments of the invention, the composition comprises a human glioma cell. Optionally, the composition comprises at least 2, 3, or 4 single guide RNAs. In certain embodiments of the invention, the single guide RNA(s) of the composition comprises nucleotides that are complementary to a CpG island region in SEQ ID NO: 1. For example, in some embodiments of the invention, the single guide RNA comprises nucleotides that are complementary to at least one CpG site 1-98 in SEQ ID NO: 1. In illustrative working embodiments of the invention, the single guide RNA(s) comprises at least one sequence selected from:

sgRNA I
(5′-GGTGCGCACCGTTTGCGACT-3′
(SEQ ID NO: 2), PAM = TGG (SEQ ID NO: 3));
sgRNA II
(5′-AGGCGCCCTCACTTCGCCGT-3′
(SEQ ID NO: 4), PAM = CGG (SEQ ID NO: 5));
sgRNA III
(5′-CTTTGCGTCCCGACGCCCGC-3′
(SEQ ID NO: 6), PAM = AGG (SEQ ID NO: 7));
and
sgRNA IV
(5′-AGGGCATGCGCCGACCCGGT-3′
(SEQ ID NO: 8), PAM = CGG (SEQ ID NO: (9).

Compositions of the invention can be adapted for use with certain delivery systems including viral, non-polymeric, and polymeric vectors that have been used in glioblastoma multiforme (GBM) gene therapy (see, e.g., Caffery et al, Nanomaterials 2019, 9 (1), 105). In certain embodiments of the invention, the composition comprises an expression vector, wherein the fusion protein is encoded by the expression vector. Optionally, the expression vector comprises a lentiviral, adenoviral or adeno-associated viral vector. In some embodiments of the invention, the composition comprises further agents such as a lipid and/or a pharmaceutical excipient. For example, in some embodiments of the invention, the CRISPR ribonucleotide complex is disposed within a lipid nanoparticle, wherein the lipid nanoparticle is coupled to a blood-brain barrier permeability enhancing agent and/or a polypeptide that specifically binds glioma cells.

Once a sgRNA complement is selected (i.e. a minimum number of sgRNAs required to achieve MGMT methylation and associated chemosensitivity/improved survival in animal studies), we can package the dCas9/CRISPR-based MGMT targeting system in an appropriate clinical delivery system which achieves the following goals: 1) maximizes patient safety; 2) enhances specificity to the genetic target of interest; 3) optimizes specific delivery to the tumor cells; 4) achieves sufficient penetration of the blood-brain barrier. To this end, we will select a vector that meets all of the above the criteria within an appropriate clinical safety margin.

Illustrative strategies for the clinical delivery system include (but are not limited to) the following: Lipid nanoparticle-based delivery of ribonucleoprotein complexes, and/or lentivirus-based vectors, and/or adenovirus-based vectors. The target specificity of lipid nanoparticles can be further enhanced by attachment of receptors or ligands which are unique to glioma cells; nanoparticle delivery to tumor cells can also be enhanced via conjugation with specific blood-brain barrier permeability-enhancing agents. Clinical delivery routes for the dCas9/CRISPR-based methylation system include (but are not limited to) the following: Intratumoral infusion and/or convection-enhanced delivery, or injection/saturation of the tumor resection cavity margins, or via overlay of a time-released biocompatible gel or wafer system perioperatively, immediately following tumor resection.

Embodiments of the invention also include methods of modulating the physiological response of a mammalian glioma cell to a chemotherapeutic agent. Typically these methods comprise combining a mammalian glioma cell with a composition of the invention so that the CRISPR ribonucleotide methylation complex methylates a plurality of CpG dinucleotide sites in a O6-methyguanine DNA methyltransferase polynucleotide in the genome of the mammalian glioma cell such that the physiological response of the mammalian glioma cell to the chemotherapeutic agent is modulated. In typical methods of the invention, the chemotherapeutic agent is temozolomide. In certain embodiments of the invention, the glioma is a glioblastoma such as a Glioblastoma multiforme. In some methods of the invention, the CRISPR ribonucleotide complex is disposed within a lipid nanoparticle, wherein the lipid nanoparticle is coupled to a blood-brain barrier permeability enhancing agent and/or a polypeptide that specifically binds glioma cells.

The sgRNAs (four guide RNAs total) utilized in our illustrative working embodiments of a dCas9/DNMT3A-CD CRISPR-based system were specifically designed to target selected MGMT promoter regions methylation by the DNMT3A methyltransferase catalytic domain. Bisulfite DNA sequencing allowed for the direct confirmation of methylation status in the target regions identified as having functional significance. Sequences for sgRNAs were determined using the Broad Institute Genetic Perturbation Platform and the following input sequence mapping to the MGMT region of interest: TGCCCCTCGGCCCCGCCCCCGCGCCCCGGATATGCTGGGACAGCCCGCGC CCCTAGAACGCTTTGCGTCCCGACGCCCGCAGGTCCTCGCGGTGCGCACC GTTTGCGACTTGGTGAGTGTCTGGGTCGCCTCGCTCCCGGAAGAGTGCGG AGCTCTCCCTCGGGACGGTGGCAGCCTCGAGTGGTCCTGCAGGCGCCCTC ACTTCGCCGTCGGGTGTGGGGCCGCCCTGACCCCCACCCATCCCGGGCGA (SEQ ID NO: 1). Sequences for sgRNA I-IV utilized in illustrative working embodiments are as follows:

sgRNA I
(5′-GGTGCGCACCGTTTGCGACT-3′
(SEQ ID NO: 2), PAM = TGG (SEQ ID NO: 3));
sgRNA II
(5′-AGGCGCCCTCACTTCGCCGT-3′
(SEQ ID NO: 4), PAM = CGG (SEQ ID NO: 5));
sgRNA III
(5′-CTTTGCGTCCCGACGCCCGC-3′
(SEQ ID NO: 6), PAM = AGG (SEQ ID NO: 7));
and
sgRNA IV
(5′-AGGGCATGCGCCGACCCGGT-3′
(SEQ ID NO: 8), PAM = CGG (SEQ ID NO: 9).
Scrambled guide RNA (scRNA)
(5′-GTATTACTGATATTGGTGGG-3′
(SEQ ID NO: 10)

Examples of CRISPR ribonucleoprotein complexes, the CRISPR-associated RNA and protein components, and CRISPR-associated systems are disclosed in the following references: Collingwood, M. A., Jacobi, A. M., Rettig, G. R., Schubert, M. S., and Behlke, M. A., “CRISPR-BASED COMPOSITIONS AND METHOD OF USE,” U.S. patent application Ser. No. 14/975,709, filed Dec. 18, 2015, published now as U.S. Patent Application Publication No. US2016/0177304A1 on Jun. 23, 2016 and issued as U.S. Pat. No. 9,840,702 on Dec. 12, 2017; and Behlke, M. A. et al. “CRISPR/CPF1 SYSTEMS AND METHODS,” U.S. patent application Ser. No. 15/821,736, filed Nov. 22, 2017, and U.S. Patent Application Publication No. 20190032131, the contents of which are hereby incorporated by reference herein in their entirety. Further aspects and embodiments of the invention are discussed below.

As discussed below, we employed, CRISPR-based mechanism to target MGMT, consisting of a chimeric fusion of deactivated Cas 9 (dCas9) with an epigenetic editor, DNA methyltransferase 3A (DNMT3A), catalyzing CpG methylation in a targeted fashion (16). We specifically employed the DNMT3A catalytic domain only (DNMT3A-CD) (17). This approach has multiple advantages in comparison to other gene silencing techniques: 1) Specificity, due to single guide RNA (sgRNA) interactions with dCas9, facilitating target gene recognition; and 2) reversibility and relative safety, due to preservation of the underlying target DNA sequence, resulting from deactivation of Cas9 endonuclease activity (avoiding permanent cleavage of gene sequences) and due to modification at the epigenetic level only (methylation). Furthermore, we used multiple sgRNAs to methylate a wider range of target sequences, encompassing the promoter and enhancer region, thereby enhancing effects on MGMT transcription. Our target regions also included: 1) Differentially Methylated Region 2 (DMR2), most highly associated with MGMT mRNA suppression (18); and 2) Methylation-Specific Polymerase Chain Reaction (MSP) region, conventionally used clinically to determine MGMT methylation status, located within DMR2 (2, 19).

### Methods

Cell Culture and Treatments. LN18 cells (ATCC, Cat #CRL-2610) were grown in standard conditions (DMEM cell culture medium, 10% fetal bovine serum and penicillin/streptomycin). TMZ (Santa Cruz Biotechnology Cat #85622-93-1) was dissolved in DMSO.

Plasmids and Lentiviral Transduction. dCas9-DNMT3A catalytic domain plasmids were constructed by Pflueger et al. (Addgene, Cat #100936). Cas9 plasmids were obtained from Addgene (Cat #108100). We designed sgRNA sequences using the Broad Institute Genetic Perturbation Platform.

All sgRNA constructs were mounted on lentivirus-compatible plasmids (Vector Builder). Plasmids were packaged with pMD2.G VSV-G envelope plasmid (Addgene, Cat #12259), pCMVR8.74 packaging plasmid (Addgene, Cat #22036), and X-tremeGENE HP DNA Transfection Reagent (MilliporeSigma, Cat #XTGHP-RO) in HEK293T cells cultured in DMEM; virus was harvested after 48 hours. LN18 cells were transfected with lentivirus-containing media and culture media in a 1:3 ratio including polybrene (1.0 μg/mL) for 48 hours, with 24-hour recovery in DMEM, prior to antibiotic selection.

Bisulfite Sequencing. DNA was isolated using DNEasy Blood & Tissue Kit (Qiagen, Catalog #69506). Bisulfite conversion was accomplished using EZ DNA Methylation-Gold (Zymo Research, Catalog #D5005) per kit protocol. Nested PCR primers were used as follows:

Region 1: First PCR primer pair F4/R4; second PCR pair F5/R4.

Region 2: First PCR primer pair F1/R1; second PCR pair F1/SeqR.

Sequencing reactions were performed using BigDye Terminator v3.1 Cycle Sequencing Kit (ThermoFisher, Cat #433750) with sequencing primers (R4 primer=Region 1; SeqR primer=Region 2); samples were purified by PCR Clean-Up Performa Spin Columns (EdgeBio Cat #13266) and submitted for Sanger sequencing analysis (Laragen, Culver City, CA).

TA Cloning. Sodium bisulfite-treated genomic DNA underwent nested PCR as above, for either Region 1 or 2. Resultant amplicons (2nd PCR products) were ligated with plasmid vector using the TA cloning kit (New England BioLabs, Cat #E1203S), used to transform DH5a competent E. coli cells (Invitrogen, Cat #18258012) by standard methods; clonal plasmids were isolated by PureLink™ HiPure Plasmid Miniprep kit (Invitrogen, Cat #K210003). DNA plasmid clones were sequenced per standard sequencing protocols as above.

RT-qPCR. RT was performed using SuperScript™ II Reverse Transcriptase (Invitrogen, Cat #REF100004925), followed by qPCR using standard protocols, with Roche FastStart Universal Master SybrGreen (Rox) (Sigma Aldrich, Cat #4913850001); annealing temperature: 550C; cycle: 30.

Methylation Specific PCR. Refer to previous publication for methods (20).

Immunoblotting. Western blot was performed by standard protocols using primary antibodies: anti-HA, rabbit, (1:1000, Sigma, Cat #H6908-100 mL); anti-MGMT, mouse (1:1000, ThermoFisher, Cat #35-7000); GAPDH, mouse (1:2000, Proteintech Cat #60004-I-Ig).

Cell Survival Assays. MTT (3-(4,5-dimethyl-2-thiazolyl)-2,5-diphenyl-2H-tetrazolium bromide) assays were performed as previously described (21-23). Briefly, a uniform number of cells (2300 or 4600 per plate) were cultured in 24-well plates for 5 days with TMZ (100 μM or 250 μM) or DMSO control treatment, exposed to pre-mixed MTT solution (0.5 mg/mL in culture media), and incubated at 37° C. for 1 hour. Formazan product was extracted by cell lysis with DMSO (300 μL) and measured (560 nm absorbance with background subtraction of 660 nm). Clonogenic assays were conducted as previously described (21-23). Cells (250 or 350 per well) were seeded in 60-mm plates. After overnight incubation, TMZ/DMSO was added and replaced after 6 days. Following 12 days of total treatment, cells were washed in phosphate-buffered saline (PBS), fixed in 100% methanol, and stained with 0.5% crystal violet/25% methanol solution.

Statistical Methods for MTT and Clonogenic Assays. Data were analyzed in Prism 9 via a student's t-test or analysis of variance (ANOVA) where appropriate, and ANOVA post hoc analyses were performed using Tukey's HSD test for multiple comparisons.

Differential Methylation Analysis and Transcriptomic Analysis. Genome-wide methylation and transcription profiling was achieved via the Illumina platform EPIC 850K methylation array. Briefly, genomic data (IDAT files) were imported into R (24) and processed via the minfi package for the generation of methylation M-values (25). Custom scripts were written to determine variance (standard deviation) for each probe for unsupervised hierarchical clustering, while supervised hierarchical clustering was achieved by fitting the data to a linear model and evaluating it via empirical Bayes for differential methylation (26). Expression (RNA transcription) was determined by aligning bulk RNA-Seq data to the genome via minimap2 and counting genes via HTSeq, which were then analyzed for differential expression using DESeq2 (27). Theoretical sgRNA “hits” were determined using the Off-Spotter platform (28).

### Results

Selection of unmethylated MGMT cellular background, epigenetic editor, sgRNA construct design, and expression verification.

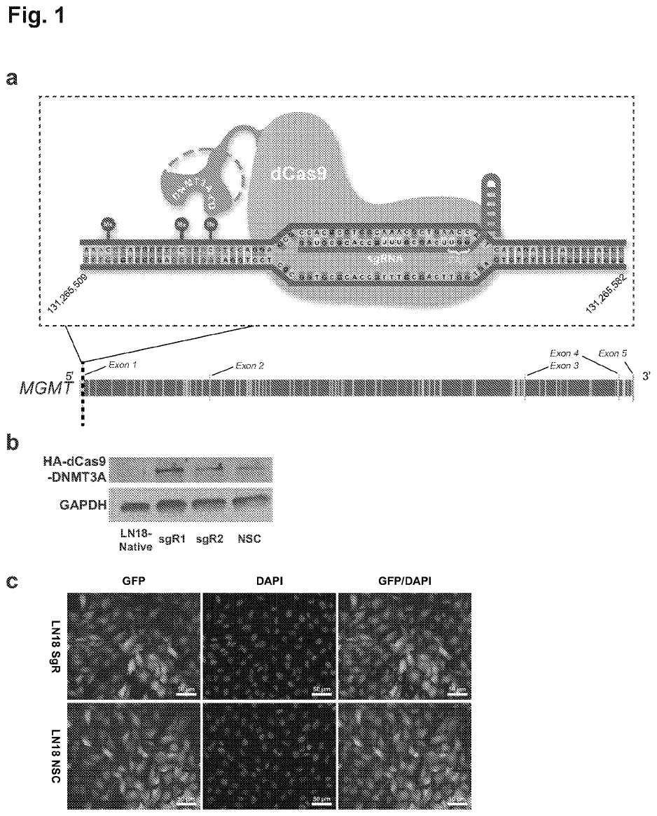

After screening multiple glioma cell lines with MSP and bisulfite sequencing (BiSEQ), we selected LN18 human glioma cells, given the unmethylated MGMT status within target regions of interest and high levels of de novo MGMT expression. LN18 exhibits high TMZ EC50 values (400 μM) amongst glioma cell lines with chemoresistance reported over time (29). For the effector enzyme, we selected the de novo methylator DNMT3A catalytic domain fused to dCas9 via a flexible linker plus HA tag (17), comprising a smaller plasmid construct more amenable to lentiviral delivery methods (FIG. 1a).

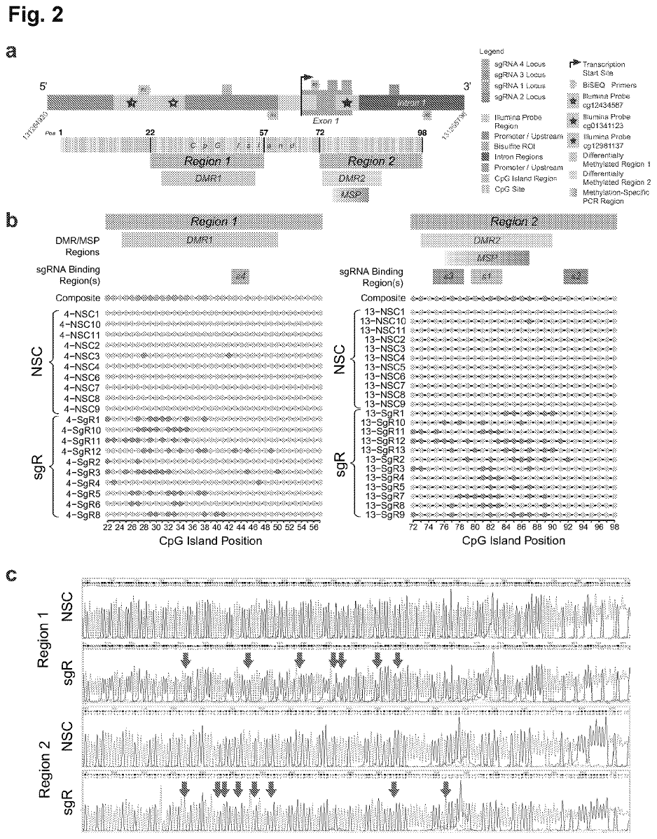

Given reported enhanced efficiency of dCas9-mediated methylation using multiple sgRNAs broadening target regions (16, 17, 30-33), we designed four sgRNAs with specific homology to MGMT regions encompassing promoter, enhancer and exon 1 regions, within a GFP-tagged lentiviral plasmid (FIG. 2a). As a negative control, we established LN18 cells expressing dCas9-DNMT3A-CD plus NSC (non-specific, scrambled sgRNA), bearing no exact sequence homology to any mammalian genomic regions. We verified expression of HA-tagged dCas9-DNMT3A-CD via western blot analysis of sgRNA-transfected cells and NSC-transfected cells, relative to baseline native LN18 cells (FIG. 1b). Fluorescent microscopy confirmed sgRNA and NSC expression (FIG. 1c), showing efficient lentiviral transfection efficiency (over 90% cells exhibiting GFP positivity).

BiSEQ confirms targeted MGMT methylation via dCas9/CRISPR system, suggesting methylation hotspot locations and possible minimum radius of methylation.

Using BiSEQ, the gold standard for methylation status confirmation of individual CpG sites, we analyzed the following targets separately using TA cloning methods: 1) “Region 1,” encompassing sgRNA4, core/minimal promoter, and promoter regions; 2) “Region 2,” comprised of exon 1, intron 1 and enhancer in proximity to sgRNA1, 2, and 3 (FIG. 2a, 2b). Due to aforementioned key components which influence gene transcription, we selected these target regions, which also included Differentially Methylated Regions (DMR1 and DMR2) and the MSP region (18, 19). Representative chromatograms are shown (FIG. 2c), comparing NSC vs. SgR clones; blue arrows indicate methylated CpG sites (retained cytosines at CpG sites, “CG”) vs. unmethylated CpG sites (converted to thymine, “TG”).

The “composite” row of the schematic (FIG. 2b) illustrates relative densities of methylated CpG sites; red shading intensity indicates methylation frequency amongst composite clones. Amongst Region 1 clones, there is apparent asymmetry with respect to methylation occurrence: The preponderance of methylated sites were located in the 5′ upstream region relative to sgRNA4, at CpG 22-38 (DMR1 and promoter region) but minimally noted in the partially overlapping region with the core promoter (CpG 50 and downstream to end of Region 1), which is downstream from sgRNA4. For Region 2, which encompassed sgRNA3, sgRNA1, and sgRNA2 target sequences, methylated CpG sites clustered toward the center of the amplicon, upstream of sgRNA2, within DMR2 and MSP regions, with greatest density and frequency at CpG 77, 81-89. Within Region 1, methylation appears to extend to a 20-bp radius upstream from sgRNA4, to the upstream 5′ limit of the amplicon (CpG 22). It is unknown whether methylation extends further upstream beyond CpG 22, as we did not assay this region here. Given close proximities of multiple sgRNA target sequences within Region 2 (sgRNA 1, 2, 3), methylation radius cannot be reliably ascertained in Region 2.

CRISPR-based MGMT methylation is sufficient to reduce MGMT expression and enhance chemosensitivity.

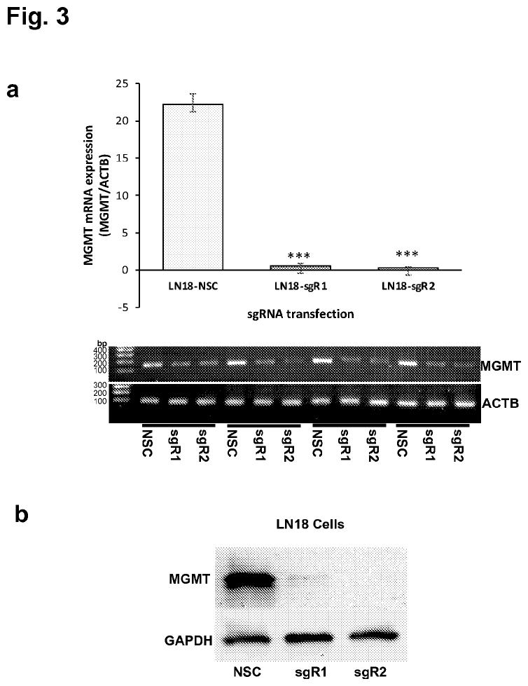

Using polyclonal populations of cell lines stably transfected with MGMT-specific sgRNAs (SgR) or NSC, RT-qPCR was performed, revealing significant downregulation in MGMT mRNA expression (p<0.001, FIG. 3a). Gel electrophoresis of PCR end products is also shown (FIG. 3b). Protein lysates from the same cell lines were analyzed by western blot, revealing marked downregulation of MGMT protein expression in the context of CRISPR-based methylation (FIG. 3b).

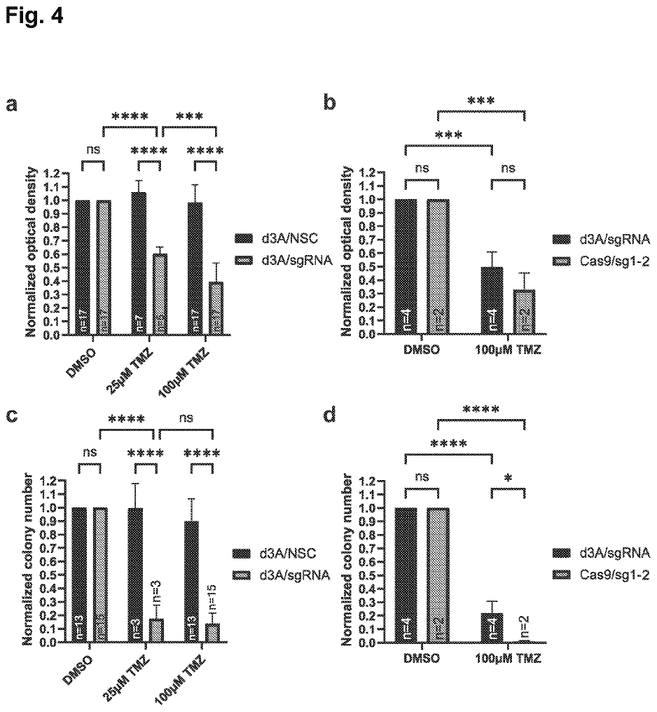

We investigated whether CRISPR-mediated epigenetic conversion was sufficient to enhance TMZ chemosensitivity, using MTT and clonogenic survival assays, both as previously described (21-23). We compared survival in SgR and NSC cells treated with DMSO (vehicle), 25 μM TMZ, or 100 μM TMZ using two-factor ANOVA. ANOVA analysis of MTT results revealed main effects of sgRNA treatment (F (1, 74)=213.3, p<0.0001), TMZ treatment (F (2,74)=93.52, p<0.0001), as well as an sgRNA/TMZ interaction (F (2, 74)=86.92, p<0.0001) (FIG. 4a). Clonogenic assays yielded analogous results between low-dose TMZ-treated cells and DMSO controls (ANOVA, TMZ treatment F (2, 56)=186.2, p<0.0001) (FIG. 4c). A post hoc Tukey test (p<0.0001) showed enhanced sensitization to TMZ occurred at concentrations as low as 25 μM (FIG. 4c).

To compare direct Cas9 endonuclease disruption of MGMT vs. deactivated Cas9-based methylation effects, we generated Cas9-mediated MGMT knockout LN18 cells. Extent of chemosensitization is comparable between Cas9-mediated knockout lines vs. dCas9-DNMT3A-CD-mediated methylation when evaluated via MTT (ANOVA, F (1, 8)=2.843, p=0.1303), but a difference was observed via clonogenic assays (F (1, 8)=9.587, p=0.0147). Both assays, however, demonstrate clear TMZ sensitivity due to dCas9-DNMT3A-CD-mediated methylation (FIG. 4b, 4d). Essentially no TMZ sensitivity was observed in LN18 dCas9-DNMT3A cells expressing the NSC negative control construct. This indicates that epigenetic conversion is a sufficient, viable alternative strategy to enhance chemotherapeutic response, obviating the need for permanent target gene cleavage.

Validation of dCas9/CRISPR-based target specificity: Genome-wide vs. transcriptome-wide correlative analysis.

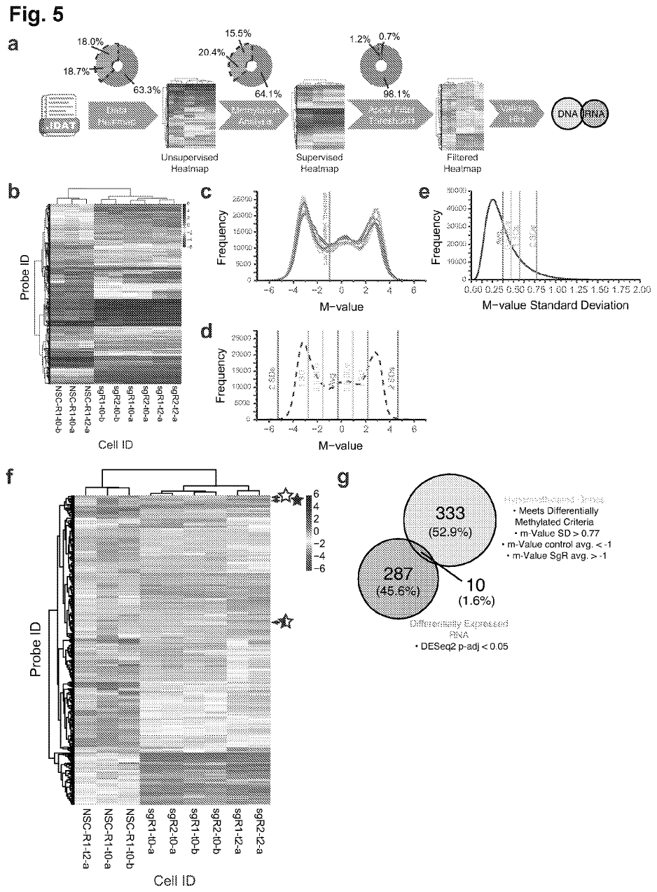

To determine on-target and off-target effects of dCas9/CRISPR-based methylation, we performed genome-wide analysis of LN18 cells expressing the full dCas9-DNMT3A-CD/sgRNA system vs. dCas9-DNMT3A-CD/NSC using the Illumina EPIC 850K methylation array, followed by transcriptomic analysis via bulk RNA-Seq. Probes with the highest M-value variances (2.5 SDs greater than the average M-value SD) were used to generate an unsupervised hierarchical heatmap (total 21,278 probes), plotted by Illumina probe and LN18 cell type (SgR vs. NSC). This necessary step demonstrated that samples of concordant cell type segregated accordingly (NSC samples clustered together and SgR samples likewise clustered together) (FIG. 5b). Supervised hierarchical clustering was performed sequentially, identifying genes with the greatest difference in methylation state following dCas9/CRISPR-mediated methylation (baseline unmethylated in control NSC cells but methylated in SgR cells). A total of 333 unique genes were identified, including three probes within the MGMT gene (FIG. 5g): cg12434587 (open star) and cg01341123 (half-closed star), both in proximity to sgRNA4, and cg12981137 (closed star), in proximity to sgRNA1, 2, and 3 loci (FIG. 2a), providing additional confirmation of on-target effects.

To elucidate effects on transcriptomic changes, we performed Bulk RNA-Seq analysis on the same LN18 glioma cell lines. The 333 unique genes identified by secondary supervised hierarchical clustering were cross-referenced with bulk RNA-Seq differential expression data, yielding 10 total gene hits, including MGMT as the top probe hit, serving as on-target confirmation. Subsequently, we used the Off-Spotter program (28) to blast sgRNA 1, 2, 3, 4 sequences for off-target prediction hits. Of the 10 genes that emerged from the 3-part analysis, namely, 1) DNA differential methylation, 2) RNA differential expression, 3) Off-spotter intersection, MGMT emerged as the singular gene hit fitting all criteria: the only hits emerging were MGMT probes. The 9 additional “off target” gene probe hits had Off-Spotter hits hundreds of thousands of bases away from the probe that emerged from analysis. One exception was FAM84A, but RNA expression was incongruent with DNA methylation (i.e., increased DNA methylation but with increased RNA transcription). From basic interrogation of Off-Spotter hits within 1000 bases of the Illumina probe, none of the results fit the aforementioned DNA/RNA criteria. Our findings suggest dCas9/CRISPR-based methylation appears specific for MGMT with minimal off-target effects.

The disclosure herein provides proof-of-principle evidence that the dCas9-DNMT3A-CD/CRISPR methylation system is sufficient for targeted methylation at a high frequency and density within the MGMT promoter and enhancer regions (including DMR1, DMR2 and MSP). This methylation is likewise sufficient for MGMT downregulation and chemosensitization, reflected by significant reductions of tumor cell survival in vitro. Correlative analyses of genomic and transcriptomic changes provided initial validation of target specificity, with no definitive off-target effects identified. Supervised hierarchical clustering of Illumina methylation data identified three differentially methylated probes within MGMT: Two probes (cg12434587 and cg12981137) were congruent with prior reports in GBM patient samples using the MGMT-STP27 logistic regression model (34), localized to the promoter, correlating with patient outcomes; the third probe (cg01341123), upstream to Region 1, has not been previously reported as a survival correlate. Future studies can confirm epigenetic and clinical significance of this upstream region via clonal analysis. The regression model noted CpG sites in proximity to TSS and on the far 5′ and 3′ ends of the CpG island did not correlate with overall survival (34). We examined the TSS region and far 3′ end, outside the regions of high methylation frequency/density, suggesting methylation in these areas is not required for MGMT suppression and chemosensitization.

Given proximity of multiple sgRNAs in Region 2, it is unknown whether they equally or hierarchically influence methylation patterns. Future studies can elucidate effects of a singular sgRNA on CpG cluster methylation, i.e. whether an upstream methylation propensity truly exists, relative to sgRNA target locus. The poorly defined methylation radius in Region 2 can be clarified with this method. The dCas9-DNMT3A-CD/CRISPR system can be conveniently harnessed to probe relative clinical significance of CpG site methylation throughout the CpG island, effects on gene transcription, and elucidate possible methylation interdependence between CpG sites. For future clinical application, the minimum complement of sgRNA payload required to achieve the current methylation patterns should be determined.

We are optimizing conditions for CRISPR-based methylation in patient-derived gliomasphere cell lines and ex vivo xenograft studies. LN18 glioma cells provided an appropriate genetic landscape for current proof-of-principle studies, but our confirmed lack of intracranial tumor engraftment in vivo, corroborated by previous attempts by others (35) necessitates using alternative cellular backgrounds.

Future translational studies of CRISPR-based methylation can address the following anticipated challenges: Selection/optimization of delivery vehicle (e.g., viral vectors vs. nanoparticles, and systemic vs. intratumoral delivery); payload definition (e.g., ribonucleoprotein complex of dCas9 and target sgRNAs); physiologic obstacles impacting optimal delivery (blood brain barrier impedance/penetration, solid tumor context preventing uniform penetration, and engineering mechanisms to achieve specificity for target cells). In summary, the current approach provides the foundation for preclinical and translational endeavors using modified dCas9/CRISPR-based epigenetic editing in the malignant glioma context, achieving chemosensitivity within a theoretically reasonable safety profile.

Methods and materials in the field of the invention are disclosed, for example in US Patent Application Publication Nos.: 20200362355, 20200048606, 20200000851 and 20190388469, as well as literature references such as: Functional CRISPR dissection of gene networks controlling human regulatory T cell identity. Schumann et al. Nat Immunol (2020); CRISPR screen in regulatory T cells reveals modulators of Foxp3; Cortez et al., Nature 2020, 29 Apr.; Pooled Knockin Targeting for Genome Engineering of Cellular Immunotherapies. Roth et al., Cell 2020 Apr. 30; Polymer-stabilized Cas9 nanoparticles and modified repair templates increase genome editing efficiency. Nguyen et al., Nat Biotechnol. 2019 Dec. 9; Landscape of stimulation-responsive chromatin across diverse human immune cells; Calderon et al., Nat Genet. 2019 Sep. 30; Large dataset enables prediction of repair after CRISPR-Cas9 editing in primary T cells; Leenay et al., Nat Biotechnol. 2019 September; 37 (9): 1034-1037; A large CRISPR-induced bystander mutation causes immune dysregulation; Simeonov et al., Commun Biol. 2019 Feb. 18:2:70; Genome-wide CRISPR Screens in Primary Human T Cells Reveal Key Regulators of Immune Function. Shifrut et al., Cell 2018 December; 175:1-14; Reprogramming human T cell function and specificity with non-viral genome targeting; Roth et al., Nature. 2018 July; 559 (7714): 405-409; “T-bet”-ing on autoimmunity variants. Nguyen et al., PLOS Genetics. 13 (9); e1006924 (2017); Discovery of stimulation-responsive immune enhancers with CRISPR activation; Simeonov et al., Nature. 549; 111-115 (2017); A Cas9 Ribonucleoprotein Platform for Functional Genetic Studies of HIV-Host Interactions in Primary Human Cells. Hulquist et al., Cell Reports. 17; 138-1452 (2016); and Generation of Knock-in Primary Human T Cells Using Cas9 Ribonucleoproteins Schumamn et al., PNAS. (2015), the contents of all of which are incorporated herein by reference.

### REFERENCES

1. Ceccarelli M, Barthel F P, Malta T M, et al (2016) Molecular Profiling Reveals Biologically Discrete Subsets and Pathways of Progression in Diffuse Glioma. Cell 164:550-563.
2. Hegi M E, Diserens A-C, Gorlia T, et al (2005) MGMT gene silencing and benefit from temozolomide in glioblastoma. N Engl J Med 352:997-1003.
3. Hegi M E, Liu L, Herman J G, et al (2008) Correlation of O6-methylguanine methyltransferase (MGMT) promoter methylation with clinical outcomes in glioblastoma and clinical strategies to modulate MGMT activity. J Clin Oncol 26:4189-4199.
4. Ostrom Q T, Gittleman H, Liao P, et al (2017) CBTRUS Statistical Report: Primary brain and other central nervous system tumors diagnosed in the United States in 2010-2014. Neuro Oncol 19: v1-v88.
5. Reifenberger G, Hentschel B, Felsberg J, et al (2012) Predictive impact of MGMT promoter methylation in glioblastoma of the elderly. Int J Cancer 131:1342-1350.
6. Stupp R, Mason W P, van den Bent M J, et al (2005) Radiotherapy plus Concomitant and Adjuvant Temozolomide for Glioblastoma. N Engl J Med 352:987-996.
7. Stupp R, Hegi M E, Gilbert M R, Chakravarti A (2007) Chemoradiotherapy in malignant glioma: standard of care and future directions. J Clin Oncol 25:4127-4136.
8. Wick W, Meisner C, Hentschel B, et al (2013) Prognostic or predictive value of MGMT promoter methylation in gliomas depends on IDH1 mutation. Neurology 81:1515-1522.
9. Brandes A A, Franceschi E, Tosoni A, et al (2009) Temozolomide concomitant and adjuvant to radiotherapy in elderly patients with glioblastoma: correlation with MGMT promoter methylation status. Cancer 115:3512-3518.
10. Malmström A, Grønberg B H, Marosi C, et al (2012) Temozolomide versus standard 6-week radiotherapy versus hypofractionated radiotherapy in patients older than 60 years with glioblastoma: the Nordic randomised, phase 3 trial. Lancet Oncol 13:916-926.
11. Rivera A L, Pelloski C E, Gilbert M R, et al (2010) MGMT promoter methylation is predictive of response to radiotherapy and prognostic in the absence of adjuvant alkylating chemotherapy for glioblastoma. Neuro Oncol 12:116-121.
12. Wiestler B, Claus R, Hartlieb S A, et al (2013) Malignant astrocytomas of elderly patients lack favorable molecular markers: an analysis of the NOA-08 study collective. Neuro Oncol 15:1017-1026.
13. Quinn J A, Jiang S X, Reardon D A, et al (2009) Phase I trial of temozolomide plus O6-benzylguanine 5-day regimen with recurrent malignant glioma. Neuro Oncol 11:556-560.
14. Schilsky R L, Dolan M E, Bertucci D, et al (2000) Phase I clinical and pharmacological study of O6-benzylguanine followed by carmustine in patients with advanced cancer. Clin Cancer Res 6:3025-3031
15. Gilbert M R, Wang M, Aldape K D, et al (2013) Dose-Dense Temozolomide for Newly Diagnosed Glioblastoma: A Randomized Phase III Clinical Trial. JCO 31:4085-4091.
16. Liu X S, Wu H, Ji X, et al (2016) Editing DNA Methylation in the Mammalian Genome. Cell 167:233-247.e17.
17. Pflueger C, Tan D, Swain T, et al (2018) A modular dCas9-SunTag DNMT3A epigenome editing system overcomes pervasive off-target activity of direct fusion dCas9-DNMT3A constructs. Genome Res 28:1193-1206.
18. Malley D S, Hamoudi R A, Kocialkowski S, et al (2011) A distinct region of the MGMT CpG island critical for transcriptional regulation is preferentially methylated in glioblastoma cells and xenografts. Acta Neuropathol 121:651-661.
19. Lalezari S, Chou A P, Tran A, et al (2013) Combined analysis of O6-methylguanine-DNA methyltransferase protein expression and promoter methylation provides optimized prognostication of glioblastoma outcome. Neuro Oncol 15:370-381.
20. Lalezari S, Chou A P, Tran A, et al (2013) Combined analysis of O6-methylguanine-DNA methyltransferase protein expression and promoter methylation provides optimized prognostication of glioblastoma outcome. Neuro Oncol 15:370-381.
21. Franken N A P, Rodermond H M, Stap J, et al (2006) Clonogenic assay of cells in vitro. Nat Protoc 1:2315-2319.
22. Hermisson M, Klumpp A, Wick W, et al (2006) O6-methylguanine DNA methyltransferase and p53 status predict temozolomide sensitivity in human malignant glioma cells. Journal of Neurochemistry 96:766-776.
23. Remington M, Chtchetinin J, Ancheta K, et al (2009) The L84F polymorphic variant of human O6-methylguanine-DNA methyltransferase alters stability in U87MG glioma cells but not temozolomide sensitivity. Neuro Oncol 11:22-32.
24. R Core Team (2014) R: A language and environment for statistical computing. R Foundation for Statistical Computing, Vienna, Austria.
25. Aryee M J, Jaffe A E, Corrada-Bravo H, et al (2014) Minfi: a flexible and comprehensive Bioconductor package for the analysis of Infinium DNA methylation microarrays. Bioinformatics 30:1363-1369.
26. Ritchie M E, Phipson B, Wu D, et al (2015) limma powers differential expression analyses for RNA-sequencing and microarray studies. Nucleic Acids Research 43: e47.
27. Love M I, Huber W, Anders S (2014) Moderated estimation of fold change and dispersion for RNA-seq data with DESeq2. Genome Biology 15:550.
28. Pliatsika V, Rigoutsos I (2015) “Off-Spotter”: very fast and exhaustive enumeration of genomic lookalikes for designing CRISPR/Cas guide RNAs. Biology Direct 10:4.
29. Happold C, Roth P, Wick W, et al (2012) Distinct molecular mechanisms of acquired resistance to temozolomide in glioblastoma cells. Journal of Neurochemistry 122:444-455.
30. Amabile A, Migliara A, Capasso P, et al (2016) Inheritable Silencing of Endogenous Genes by Hit-and-Run Targeted Epigenetic Editing. Cell 167:219-232.e14.
31. McDonald J I, Celik H, Rois L E, et al (2016) Reprogrammable CRISPR/Cas9-based system for inducing site-specific DNA methylation. Biol Open 5:866-874.
32. Stepper P, Kungulovski G, Jurkowska R Z, et al (2017) Efficient targeted DNA methylation with chimeric dCas9-Dnmt3a-Dnmt3L methyltransferase. Nucleic Acids Res 45:1703-1713.
33. Vojta A, Dobrinić P, Tadić V, et al (2016) Repurposing the CRISPR-Cas9 system for targeted DNA methylation. Nucleic Acids Res 44:5615-5628.
34. Bady P, Sciuscio D, Diserens A-C, et al (2012) MGMT methylation analysis of glioblastoma on the Infinium methylation BeadChip identifies two distinct CpG regions associated with gene silencing and outcome, yielding a prediction model for comparisons across datasets, tumor grades, and CIMP-status. Acta Neuropathol 124:547-560.
35. Takashima H, Tsuji A B, Saga T, et al (2017) Molecular imaging using an anti-human tissue factor monoclonal antibody in an orthotopic glioma xenograft model. Sci Rep 7:12341.

All publications mentioned herein (e.g., the references identified above) are incorporated by reference to disclose and describe aspects, methods and/or materials in connection with the cited publications.

Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A composition of matter comprising:
a CRISPR ribonucleotide complex; wherein:
the CRISPR ribonucleotide complex includes a fusion protein comprising a dCas9 polypeptide sequence fused with a human de novo DNA methyltransferase 3A polypeptide sequence such that the fusion protein methylates CpG sequences in the human genome and the fusion protein does not cleave DNA; and
at least one single guide RNA, wherein:
the single guide RNA comprises at least 17 nucleotides that are complementary to:
(SEQ ID NO: 1)
TGCCCCTCGGCCCCGCCCCCGCGCCCCGGATATGCTGGGACAGCCCGCGC
CCCTAGAACGCTTTGCGTCCCGACGCCCGCAGGTCCTCGCGGTGCGCACC
GTTTGCGACTTGGTGAGTGTCTGGGTCGCCTCGCTCCCGGAAGAGTGCGG
AGCTCTCCCTCGGGACGGTGGCAGCCTCGAGTGGTCCTGCAGGCGCCCTC
ACTTCGCCGTCGGGTGTGGGGCCGCCCTGACCCCCACCCATCCCGGGCG
A.

2. The composition of claim 1, wherein the single guide RNA comprises nucleotides that are complementary to a CpG island region in SEQ ID NO: 1.

3. The composition of claim 2, wherein the single guide RNA comprises nucleotides that are complementary to at least one CpG site 1-98 in SEQ ID NO: 1.

4. The composition of claim 4, wherein the single guide RNA comprises at least one sequence selected from:
sgRNA I
(5′-GGTGCGCACCGTTTGCGACT-3′
(SEQ ID NO; 2);
sgRNA II
(5′-AGGCGCCCTCACTTCGCCGT-3′
(SEQ ID NO; 4);
sgRNA III
(5′-CTTTGCGTCCCGACGCCCGC-3′
(SEQ ID NO: 6);
and
sgRNA IV
(5′-AGGGCATGCGCCGACCCGGT-3′
(SEQ ID NO: 8).

5. The composition of claim 1, wherein the composition comprises at least 2, 3, or 4 single guide RNAs.

6. The composition of claim 1, wherein:
the composition comprises an expression vector, wherein the fusion protein is encoded by the expression vector; or
the composition further comprises a glioma cell.

7. The composition of claim 6, wherein the expression vector comprises a lentiviral, adenoviral or adeno-associated viral vector.

8. The composition of claim 1, further comprising a lipid and/or a pharmaceutical excipient.

9. The composition of claim 8, wherein the CRISPR ribonucleotide complex is disposed within a lipid nanoparticle, wherein the lipid nanoparticle is coupled to a blood-brain barrier permeability enhancing agent and/or a polypeptide that specifically binds glioma cells.

10. A method of modulating the physiological response of a mammalian glioma cell to a chemotherapeutic agent; the method comprising:
combining the mammalian glioma cell with a composition of claim 1 so that the CRISPR ribonucleotide methylation complex methylates a plurality of CpG dinucleotide sites in a O6-methylguanine DNA methyltransferase polynucleotide in the genome of the mammalian glioma cell such that the physiological response of the mammalian glioma cell to the chemotherapeutic agent is modulated.

11. The method of claim 10, wherein the chemotherapeutic agent is temozolomide.

12. The method of claim 11, wherein the glioma is a glioblastoma.

13. The method of claim 12, wherein the glioma is a Glioblastoma multiforme.

14. The method of claim 13, wherein the wherein the single guide RNA comprises at least two sequences selected from:
sgRNA I
(5′-GGTGCGCACCGTTTGCGACT-3′
(SEQ ID NO; 2);
sgRNA II
(5′-AGGCGCCCTCACTTCGCCGT-3′
(SEQ ID NO; 4);
sgRNA III
(5′-CTTTGCGTCCCGACGCCCGC-3′
(SEQ ID NO: 6);
and
sgRNA IV
(5′-AGGGCATGCGCCGACCCGGT-3′
(SEQ ID NO: 8).

15. The method of claim 14, wherein the CRISPR ribonucleotide complex is disposed within a lipid nanoparticle, wherein the lipid nanoparticle is coupled to a blood-brain barrier permeability enhancing agent and/or a polypeptide that specifically binds glioma cells.


SEQUENCE LISTING (17 sequences)

SEQ ID NO: 1 - DNA, 250 bp, synthetic construct
  source 1..250: mol_type = other DNA
tgcccctcggccccgcccccgcgccccggatatgctgggacagcccgcgcccctagaacgctttgcgtcccgacgcccgcaggtcctcgcggtgcgcaccgtttgcgacttggtgagtgtctgggtcgcctcgctcccggaagagtgcggagctctccctcgggacggtggcagcctcgagtggtcctgcaggcgccctcacttcgccgtcgggtgtggggccgccctgacccccacccatcccgggcga

SEQ ID NO: 2 - DNA, 20 bp, synthetic construct
  source 1..20: mol_type = other DNA
ggtgcgcaccgtttgcgact

SEQ ID NO: 3 - skipped

SEQ ID NO: 4 - DNA, 20 bp, synthetic construct
  source 1..20: mol_type = other DNA
aggcgccctcacttcgccgt

SEQ ID NO: 5 - skipped

SEQ ID NO: 6 - DNA, 20 bp, synthetic construct
  source 1..20: mol_type = other DNA
ctttgcgtcccgacgcccgc

SEQ ID NO: 7 - skipped

SEQ ID NO: 8 - DNA, 20 bp, synthetic construct
  source 1..20: mol_type = other DNA
agggcatgcgccgacccggt

SEQ ID NO: 9 - skipped

SEQ ID NO: 10 - DNA, 20 bp, synthetic construct
  source 1..20: mol_type = other DNA
gtattactgatattggtggg

SEQ ID NO: 11 - DNA, 73 bp, synthetic construct
  source 1..73: mol_type = other DNA
  modified_base 4: mod_base = OTHER; note = Methylated nucleotide
  modified_base 14: mod_base = OTHER; note = Methylated nucleotide
  modified_base 18: mod_base = OTHER; note = Methylated nucleotide
aaacgcagggctgcgggcgtccaggagcgccacgcgtggcaaacgctgaaccactcacagacccagcggagcg

SEQ ID NO: 12 - RNA, 23 nt, synthetic construct
  source 1..23: mol_type = other RNA
ggttcagcgtttgccacgcgtgg

SEQ ID NO: 13 - DNA, 73 bp, synthetic construct
  source 1..73: mol_type = other DNA
cgctccgctgggtctgtgagtggttcagcgtttgccacgcgtggcgctcctggacgcccgcagccctgcgttt

SEQ ID NO: 14 - DNA, 224 bp, synthetic construct
  source 1..224: mol_type = other DNA
gagttttaggtggaagttgggaaggtgttgtttggtttgtattggttgaagggttatttgggttaggtgtatagggtagtggtgttgttggaggattagggttggtgtgttggtgtttagtgaggatgtgtagattgttttaggtttggtgttgttgtattgggtatgtgttgatttggttgggtgggaatattttgttttgtttgggttttgttttagttttg

SEQ ID NO: 15 - DNA, 224 bp, synthetic construct
  source 1..224: mol_type = other DNA
  modified_base 43: mod_base = OTHER; note = Methylated nucleotide
  modified_base 68: mod_base = OTHER; note = Methylated nucleotide
  modified_base 89: mod_base = OTHER; note = Methylated nucleotide
  modified_base 103: mod_base = OTHER; note = Methylated nucleotide
  modified_base 106: mod_base = OTHER; note = Methylated nucleotide
  modified_base 121: mod_base = OTHER; note = Methylated nucleotide
  modified_base 129: mod_base = OTHER; note = Methylated nucleotide
gagttttaggtggaagttgggaaggtgttgtttggtttgtatcggtcgaagggttatttgggttaggcgtatagggtagtggtgttgtcggaggattagggtcggcgtgttggtgtttagcgaggatgcgtagattgttttaggtttggtgttgttgtattgggtatgtgttgatttggttgggtgggaatattttgttttgtttgggttttgttttagttttg

SEQ ID NO: 16 - DNA, 186 bp, synthetic construct
  source 1..186: mol_type = other DNA
gtttgtgtttttagaatgttttgtgttttgatgtttgtaggtttttgtggtgtgtattgtttgtgatttggtgagtgtttgggttgttttgtttttggaagagtgtggagtttttttttgggatggtggtagttttgagtggttttgtaggtgtttttattttgttgttgggtggggggttgtttt

SEQ ID NO: 17 - DNA, 186 bp, synthetic construct
  source 1..186: mol_type = other DNA
  modified_base 35: mod_base = OTHER; note = Methylated nucleotide
  modified_base 46: mod_base = OTHER; note = Methylated nucleotide
  modified_base 48: mod_base = OTHER; note = Methylated nucleotide
  modified_base 53: mod_base = OTHER; note = Methylated nucleotide
  modified_base 58: mod_base = OTHER; note = Methylated nucleotide
  modified_base 64: mod_base = OTHER; note = Methylated nucleotide
  modified_base 106: mod_base = OTHER; note = Methylated nucleotide
  modified_base 124: mod_base = OTHER; note = Methylated nucleotide
gtttgtgtttttagaatgttttgtgttctgatgtccgtaggttttcgcggtgcgtatcgtttgcgatttggtgagtgtttgggttgttttgtttttggaagagtgcggagtttttttttgggacggtggtagttttgagtggttttgtaggtgtttttattttgttgttgggtggggggttgtttt